(19) United States
(12) Patent Application Publication
(10) Pub. No.: US 2015/0343096 A1
(43) Pub. Date: Dec. 3, 2015

(54) LIQUID PREPARATION FOR ORAL ADMINISTRATION FOR USE IN CT COLONOGRAPHY, AND COMPOSITION FOR IMAGING DIGESTIVE TRACT

(71) Applicants: Ajinomoto Co., Inc., Chuo-ku (JP); Fushimi Pharmaceutical Co., Ltd., Marugame-shi (JP)

(72) Inventors: Dennai TAKEDA, Chuo-ku (JP); Takayuki Fujiwara, Marugame-shi (JP); Nobuo Ando, Marugame-shi (JP); Tomoko Watanabe, Marugame-shi (JP); Akira Yoshino, Marugame-shi (JP)

(73) Assignees: Ajinomoto Co., Inc., Chuo-ku (JP); Fushimi Pharmaceutical Co., Ltd., Marugame-shi (JP)

(21) Appl. No.: 14/706,533

(22) Filed: May 7, 2015

Related U.S. Application Data
(63) Continuation of application No. PCT/JP2013/080254, filed on Nov. 8, 2013.

(30) Foreign Application Priority Data
Nov. 9, 2012 (JP) 2012-247927

(51) Int. Cl. A61K 49/04; A61K 47/34; A61K 47/38; A61K 9/00; A61K 9/14

(52) U.S. Cl. CPC A61K 49/0404; A61K 9/0053; A61K 9/14; A61K 47/38; A61K 47/34

(57) ABSTRACT

There are provided a liquid preparation for oral administration which contains barium that is likely to be mixed with residues and is less likely to be precipitated in the intestinal tract, and a liquid preparation for oral administration and a composition for imaging a digestive tract which contain barium and enable the reduction of the dose of an intestinal tract cleaning solution in a the intestinal tract pretreatment. That is, there are provided an orally administered liquid preparation which contains barium and one or more types of thickening agents selected from the group consisting of a natural polysaccharide and a cellulose-based polymer, in which the viscosity at a shear rate of 21.54 s⁻¹ is 100 mPa·s or greater and the viscosity at a shear rate of 464.1 s⁻¹ is 5 mPa·s to 90 mPa·s, and the particle diameter D10 which is the particle diameter when the accumulation value in a volume cumulative distribution of the barium becomes 10% is 0.30 μm to 0.80 μm and the particle diameter D90 is 1.0 μm to 10 μm; and a liquid preparation for oral administration used in CT colonography digestive tract imaging, which contains barium and a water-soluble polymer or a saline purgative agent, and is taken in an amount of 50 mL to 2000 mL all at once or in several parts over a period of time on the day before the CT colonography is performed.

### FIELD OF THE INVENTION

The present invention relates to a liquid preparation for oral administration containing barium used in a pretreatment of CT colonography, and a composition for imaging a digestive tract used in preparation of the liquid preparation for oral administration.

Priority is claimed on Japanese Patent Application No. 2012-247927, filed on Nov. 9, 2012, the content of which is incorporated herein by reference.

### DESCRIPTION OF RELATED ART

Large intestine examination (CT colonography) by computed tomography (CT) is a method of examination which is being generalized by virtue of the development of helical CT or multi-row detector CT. In the CT colonography, after the stool mass in the large intestine is washed by an intestinal tract cleaning, CT imaging is performed in a state in which the large intestine lumen is expanded with air, and image information based on the slight difference in the X-ray contrast of the large intestine tissues and air is configured and processed (for example, refer to Non-Patent Document 1).

As a method of an intestinal tract pretreatment, an intestinal tract cleaning method by an oral intestinal tract cleaning agent is widely used. In the oral intestinal tract cleaning method, as an example, a predetermined amount of powdered composition formed of an electrolyte and the like, for example, an intestinal tract cleaning liquid prepared by being dissolved in about 2 L of water is taken over 2 hours to 4 hours. In the case of using a large amount of oral intestinal tract cleaning liquid, it is possible to reduce the remaining stool, however, liquid residues increases. Therefore, a method (electronic cleansing) for electronically removing the remaining water or storage liquid in the large intestine by administering a contrast agent with the intestinal tract cleaning liquid has been used. Since the remaining water or a liquid residue-storing portion is labeled (tagged) by the contrast agent such as barium or iodine compound, and due to this, the CT value (luminance value) increases, it is possible to distinguish the polyp lesions which are not labeled with the contrast agent.

As the contrast agent used in the pretreatment for the CT colonography, barium or iodine compounds are mainly used. Barium, which is insoluble in water, is less likely to be mixed with the residues in the stomach or the digestive tract than the iodine compound, and precipitated in the remaining water in the intestinal tract in some cases. When the barium is precipitated in the remaining water, the gradient of the CT value is occurred in the remaining liquid, and thus, it is difficult to electronically remove a region in which the CT value in the remaining water in a CT image is low. Therefore, barium which is likely to be mixed with the residues and is less likely to be precipitated in the intestinal tract is required.

On the other hand, in order to use the CT colonography as a mass examination or a thorough medical examination, it is desired to increase the acceptability of a subject while maintaining the quality of the examination precision. Specifically, in the intestinal tract pretreatment method of the CT colonography in the related art, it is necessary to take a large amount of intestinal tract cleaning liquid of about 2 L for a few hours in order to sufficiently perform cleaning and removing of the intestinal tract content such as a stool mass, and thus, a heavy burden has been imposed on the subject, and it has become a problem.

In recent years, as a method for relieving the burden, a method using a liquid preparation for oral administration which is a liquid preparation for oral administration used in the digestive tract imaging of the CT colonography, contains an iodine compound, an electrolyte, and a water-soluble polymer and/or a saccharide, and is adminstered in an amount of 300 mL to 1200 mL all at once or in several parts over a period of time on the day before the CT colonography is performed in the pretreatment for the CT colonography is disclosed (for example, refer to Patent Document 1). The method is based on the finding that in order to improve the examination precision of the CT colonography, the solid residues may be eliminated from the image obtained by capturing the inside of the intestinal tract, and even in a case where the solid residues or the residues obtained by changing of the solid residues into a liquid or gel shape remain in the intestinal tract, if these residues can be excellently labeled with a contrast agent, it is possible to maintain high extraction ability in the same manner as in a case where the most part of the solid residues are completely removed, and using the method, the examination precision is not sacrificed, the time required for a pretreatment is not prolonged, the dose of the intestinal tract cleaning liquid in the intestinal tract pretreatment is reduced, and thus, the burden on a subject can be reduced.

### CITATION LIST

### Patent Document

Patent Document 1: PCT International Publication No. WO2012/046847

### Non-Patent Document

Non-Patent Document 1: Liang, J Z al., “Virtual colonoscopy: An Alternative Approach to Examination of the Entire Colon”, INNERVISION, 2001, Vol. 16, No. 10, pp. 40-44.

### SUMMARY OF THE INVENTION

### Problem to be Solved by the Invention

An object of the present invention is to provide a liquid preparation for oral administration which contains barium that is likely to be mixed with residues and is less likely to be precipitated in the intestinal tract.

In addition, another object of the present invention is to provide the liquid preparation for oral administration and the composition for imaging a digestive tract in which barium is used as a contrast agent, in which the examination precision is not sacrificed, the time required for a pretreatment is not prolonged, the dose of the intestinal tract cleaning liquid in the intestinal tract pretreatment is reduced, and thus, the burden on a subject can be reduced.

That is, the present invention provides a liquid preparation for oral administration, a composition for imaging a digestive tract, and a drug for pretreatment described below.

[1] According to a first aspect of the present invention, there is provided a liquid preparation for oral administration which contains barium sulfate and one or more types of thickening agents selected from the group consisting of a natural polysaccharide and a cellulose-based polymer, has a viscosity of 100 mPa·s or greater at a shear rate of 21.54 s⁻¹ and 5 mPa·s to 90 mPa·s at a shear rate of 464.1 s⁻¹, in which a particle diameter D10 which is a diameter when the accumulation value in a volume cumulative distribution of the barium becomes 10% is 0.30 μm to 0.80 μm and a particle diameter D90 which is a diameter when the accumulation value in a volume cumulative distribution of the barium becomes 90% is 1.0 μm to 10 μm.

[2] In the liquid preparation for oral administration according to [1], the concentration of the barium sulfate is preferably 0.15 g/mL to 0.35 g/mL.

[3] In the liquid preparation for oral administration according to [1] or [2], the viscosity at a shear rate of 21.54 s⁻¹ is preferably 350 mPa·s or greater.

[4] In the liquid preparation for oral administration according to any one of [1] to [3], the particle diameter D10 which is a diameter when the accumulation value in a volume cumulative distribution of the barium becomes 10% is preferably 0.30 μm to 0.50 μm and the particle diameter D90 which is a diameter when the accumulation value in a volume cumulative distribution of the barium becomes 90% is preferably 1.00 μm to 2.00 μm.

[5] In the liquid preparation for oral administration according to any one of [1] to [4], the thickening agent is preferably one or more types selected from the group consisting of guar gum, carrageenan, xanthan gum, fenugreek gum, pectin, pullulan, gum arabic, dextran, dextrin, polydextrose, hydroxyethyl starch, carboxymethyl cellulose and salts thereof, hydroxypropyl cellulose, gum tragacanth, sodium alginate, sodium chondroitin sulfate, locust bean gum, gellan gum, croscarmellose sodium, and crystalline cellulose-carmellose sodium.

[6] The liquid preparation for oral administration according to any one of [1] to [5] is preferably used in a CT colonography digestive tract imaging pretreatment.

[7] According to a second aspect of the present invention, there is provided a liquid preparation for oral administration used in CT colonography digestive tract imaging which contains barium and a water-soluble polymer or a saline purgative agent, and is taken in an amount of 50 mL to 2000 mL all at once or in several parts over a period of time on the day before the CT colonography is performed.

[8] The liquid preparation for oral administration according to [7] is preferably taken in an amount of 70 mL to 900 mL all at once or in several parts over a period of time on the day before the CT colonography is performed.

[9] In the liquid preparation for oral administration according to [7] or [8], the barium is preferably barium sulfate.

[10] In the liquid preparation for oral administration according to any one of [7] to [9], the water-soluble polymer is preferably one or more types selected from the group consisting of polyethylene glycol, polydextrose, dextran, dextrin, hydroxyethyl starch, gum arabic, pullulan, pectin, albumin, carboxymethyl cellulose and salts thereof, carrageenan, xanthan gum, gum tragacanth, croscarmellose sodium, and crystalline cellulose-carmellose sodium.

[11] In the liquid preparation for oral administration according to any one of [7] to [10], the osmotic pressure is preferably 200 mOsm/L to 900 mOsm/L.

[12] In the liquid preparation for oral administration according to any one of [7] to [11], one or more types of saccharides selected from the group consisting of sucrose, sorbitol, xylitol, erythritol, mannitol, trehalose, lactitol, lactulose, maltitol, palatinose, raffinose, and glycerin are preferably further contained.

[13] In the liquid preparation for oral administration according to any one of [7] to [12], the amount of the barium is preferably 8.3 mg/mL to 2000 mg/mL.

[14] In the liquid preparation for oral administration according to any one of [7] to [13], the concentration of the barium is preferably 0.10 g/mL to 0.35 g/mL.

[15] In the liquid preparation for oral administration according to [14], the viscosity at a shear rate of 21.54 s⁻¹ is preferably 350 mPa·s or greater, and the viscosity at a shear rate of 464.1 s⁻¹ is preferably 5 mPa·s to 90 mPa·s.

[16] In the liquid preparation for oral administration according to any one of [7] to [15], a particle diameter D10 which is a diameter when the accumulation value in a volume cumulative distribution of the barium becomes 10% is preferably 0.30 μm to 0.50 μm and a particle diameter D90 which is a diameter when the accumulation value in a volume cumulative distribution of the barium becomes 90% is preferably 1.00 μm to 2.00 μm.

[17] In the liquid preparation for oral administration according to any one of [7] to [16], one or more types selected from the group consisting of dimethyl polysiloxane and a digestive tract function-promoting agent are preferably further contained.

[18] According to a third aspect of the present invention, there is provided a composition for imaging a digestive tract which contains barium and a water-soluble polymer or a saline purgative agent, and is taken in an amount of 50 mL to 2000 mL all at once or in several parts over a period of time on the day before the CT colonography is performed, as an aqueous solution in which the amount of the barium is 8.3 mg/mL to 2000 mg/mL obtained by dissolving in water.

[19] The composition for imaging a digestive tract according to [18] is preferably taken in an amount of 70 mL to 900 mL all at once or in several parts over a period of time on the day before the CT colonography is performed, as the aqueous solution in which the amount of the barium is 8.3 mg/mL to 2000 mg/mL obtained by dissolving in water.

[20] In the composition for imaging a digestive tract according to [18] or [19], the barium is preferably barium sulfate.

[21] In the composition for imaging a digestive tract according to any one of [18] to [20], an electrolyte is contained such that the osmotic pressure becomes 200 mOsm/L to 900 mOsm/L when dissolved in water, and one or more types of water-soluble polymers selected from the group consisting of polyethylene glycol, polydextrose, dextran, dextrin, hydroxyethyl starch, gum arabic, pullulan, pectin, albumin, carboxymethyl cellulose and salts thereof, carrageenan, xanthan gum, gum tragacanth, croscarmellose sodium, and crystalline cellulose-carmellose sodium, or one or more types of saline purgative agents selected from the group consisting of magnesium citrate, sodium dihydrogen phosphate, and disodium hydrogen phosphate are preferably contained.

[22] In the composition for imaging a digestive tract according to any one of [18] to [21], one or more types selected from the group consisting of dimethyl polysiloxane and a digestive tract function-promoting agent are preferably further contained.

[23] In the composition for imaging a digestive tract according to any one of [18] to [22], a particle diameter D10 which is a diameter when the accumulation value in a volume cumulative distribution of the barium becomes 10% is preferably 0.30 μm to 0.50 μm and a particle diameter D90 which is a diameter when the accumulation value in a volume cumulative distribution of the barium becomes 90% is preferably 1.00 μm to 2.00 μm.

[24] The composition for imaging a digestive tract according to [23] is preferably in liquid form, and preferably has the viscosity at a shear rate of 21.54 s⁻¹ of 350 mPa·s or greater, and the viscosity at a shear rate of 464.1 s⁻¹ of 5 mPa·s to 90 mPa·s.

[25] According to a fourth aspect of the present invention, there is provided a drug for pretreatment for CT colonography which is obtained by combining barium and a water-soluble polymer or a saline purgative agent, and each of 50 mL to 2000 mL of an aqueous solution obtained by being dissolved the water-soluble polymer or the saline purgative agent in water and the barium are taken all at once or in several parts over a period of time on the day before the CT colonography is performed.

[26] In the drug for pretreatment for CT colonography according to [25], the water-soluble polymer or the saline purgative agent dissolved in water and the barium are preferably taken within 30 minutes.

[27] In the drug for pretreatment for CT colonography according to [25] or [26], the total dose of the aqueous solution on the day before the CT colonography is performed is preferably 70 mL to 900 mL.

[28] The drug for pretreatment for CT colonography according to any one of [25] to [27] is preferably a liquid preparation in which the barium is barium sulfate in which a particle diameter D10 which is a diameter when the accumulation value a volume cumulative distribution of the barium becomes 10% is 0.30 μm to 0.50 μm and a particle diameter D90 which is a diameter when the accumulation value in a volume cumulative distribution of the barium becomes 90% is 1.00 μm to 2.00 μm, and the viscosity at a shear rate of 21.54 s⁻¹ is 350 mPa·s or greater, and the viscosity at a shear rate of 464.1 s⁻¹ is 5 mPa·s to 90 mPa·s.

[29] According to a fifth aspect of the present invention, there is provided a liquid preparation for oral administration which is a liquid preparation for oral administration used in CT colonography digestive tract imaging, contains a water-soluble polymer or a saline purgative agent, and is taken with or without barium in an amount of 50 mL to 2000 mL all at once or in several parts over a period of time on the day before the CT colonography is performed.

### Effect of the Invention

In the pretreatment for the CT colonography, by using the liquid preparation for oral administration according to the first aspect of the present invention, it is possible to label remaining water or the like in the intestinal tract by barium sufficiently and uniformly to the lower portion of the large intestine.

In addition, in the pretreatment for the CT colonography, by using a liquid preparation for oral administration according to a second aspect of the present invention, a liquid preparation for oral administration according to a fifth aspect of the present invention, or a drug for pretreatment for the CT colonography according to a fourth aspect of the present invention, it is possible to significantly reduce the burden on a subject without sacrificing the quality of the examination precision. That is, since the dose per one time is sufficiently small compared to the normal capacity (2 L) of an oral intestinal tract cleaning agent used in an large intestine examination in the related art, the liquid preparation for oral administration according to the second aspect of the present invention can be taken in a short time, and thus, the burden on a subject is small. Furthermore, the number of days required for a pretreatment can be only one day before examination is performed, and it is also possible to incorporate into the examination system of hospital such that many patients can be examined simply and in a short time as a mass examination system or a thorough medical examination system.

In addition, by using the composition for imaging a digestive tract according to the third aspect of the present invention, it is possible to prepare the liquid preparation for oral administration according to the second aspect of the present invention as a formulation which is simple and has excellent feeling of administration.

### BRIEF DESCRIPTION OF THE DRAWINGS

FIG. 1 is a CT image of a supine position of a descending colon captured in Example 4.

FIG. 2 is a CT image of the supine position of the descending colon captured after taking a barium prototype liquid preparation a on the previous day, in Example 5.

FIG. 3 is a CT image of the supine position of the descending colon captured after taking a barium prototype liquid preparation b on the previous day, in Example 5.

### DETAILED DESCRIPTION OF THE INVENTION

### Liquid Preparation for Oral Administration A

A liquid preparation for oral administration (hereinafter, also referred to as “liquid preparation for oral administration A”) according to the first aspect of the present invention is a powder, contains barium sulfate having a specific particle size distribution and one or more types of thickening agents selected from the group consisting of a natural polysaccharide and a cellulose-based polymer, and is slurry having a viscosity within a specific range.

When the residues or the remaining stool in the intestinal tract are labeled with barium sulfate, performing uniform labeling is one of the conditions that lead to a good image. At that time, by making the viscosity as low as possible, the barium sulfate and the object to be labeled (residues) are efficiently mixed due to the motion of the stomach and intestines, and by making the barium sulfate particles as small as possible, precipitation of the barium sulfate can be suppressed, and due to this, the object to be labeled can be uniformly labeled. Although the conditions of particles and viscosity of the barium sulfate are shown here, by further combining other formulation characteristics, it is also possible to variously combine the conditions of the viscosity and the particle size.

A particle diameter D10 (hereinafter, simply referred to as “particle diameter D10”) which is a diameter when the accumulation value in a volume cumulative distribution of the barium sulfate contained in the liquid preparation for oral administration A according to the present invention becomes 10% is 0.30 μm to 0.80 μm and a particle diameter D90 (hereinafter, simply referred to as “particle diameter D90”) which is a diameter when the accumulation value in a volume cumulative distribution of the barium sulfate contained becomes 90% is 1.0 μm to 10 μm. In the liquid preparation for oral administration A according to the present invention, barium sulfate having a small size and a particle size distribution with a narrow distribution width is used, and thus, precipitation is less likely to occur in the remaining water or the like in the intestinal tract, and labeling is less likely to become ununiform. The barium sulfate has the particle diameter D10 of preferably 0.30 μm to 0.50 μm and more preferably 0.30 μm to 0.43 μm. In addition, the barium sulfate has the particle diameter D90 of preferably 1.0 μm to 4.0 μm and more preferably 1.0 μm to 2.0 μm. Moreover, in the present invention and the present specification, the particle size distribution of the barium sulfate means the volume cumulative distribution obtained by a laser diffraction scattering type particle size distribution measuring method.

In the liquid preparation for oral administration A according to the present invention, one or more types of thickening agents selected from the group consisting of a natural polysaccharide and a cellulose-based polymer are contained to give flow characteristics. Although the natural polysaccharide or the cellulose-based polymer is not particularly limited as long as it is edible, the natural polysaccharide or the cellulose-based polymer is preferably water-soluble. The liquid preparation for oral administration A according to the present invention may contain one type of thickening agent, or may contain two or more types of thickening agents in combination to attain the viscosity characteristics.

Examples of the natural polysaccharide include guar gum, carrageenan, xanthan gum, fenugreek gum, pectin, pullulan, gum arabic, dextran, dextrin, polydextrose, hydroxyethyl starch, gum tragacanth, sodium alginate, sodium chondroitin sulfate, locust bean gum, gellan gum, and croscarmellose sodium. In addition, examples of the cellulose-based polymer include carboxymethyl cellulose, carboxymethyl cellulose salts such as carboxymethyl cellulose sodium, hydroxypropyl cellulose, and crystalline cellulose-carmellose sodium.

The liquid preparation for oral administration A according to the present invention can be prepared by dissolving or dispersing, for example, the barium sulfate having a specific size and the thickening agent in an aqueous medium. The aqueous medium is not particularly limited as long as it is an edible aqueous medium which is inert to the barium sulfate, and the aqueous medium may be simply water, may be an oral rehydration solution, or may be a medium obtained by adding a contrast agent having barium sulfate as an effective component to a liquid oral intestinal tract cleaning agent (intestinal tract cleaning liquid) used when performing intestinal tract cleaning as a pretreatment for endoscopic examination of large intestine, barium enema examination, or the CT colonography. That is, a liquid preparation prepared by mixing the barium sulfate in the oral intestinal tract cleaning agent and, if necessary, mixing the thickening agent so as to have the specific flow characteristics can be used as the liquid preparation for oral administration A according to the present invention.

Although the oral intestinal tract cleaning agent is not particularly limited as long as it contains at least one of a water-soluble polymer and a saline purgative agent, the oral intestinal tract cleaning agent preferably includes an electrolyte such that the osmotic pressure at the time of dosing becomes 200 mOsm/L to 900 mOsm/L (preferably, 250 mOsm/L to 570 mOsm/L, more preferably 250 mOsm/L to 440 mOsm/L, and still more preferably 250 mOsm/L to 320 mOsm/L), and preferably further contains a sweetener and/or a flavoring. Specific examples can include a preparation which is obtained by combining polyethylene glycol and electrolyte (for example, “Niflec” (registered trademark) mixed internal drug (manufactured by Ajinomoto Pharmaceuticals Co., Ltd.) according to the present invention disclosed in Japanese Unexamined Patent Application, First Publication No. H1-125319), a preparation which is obtained by combining at least one water-soluble polymer selected from dextran, dextrin, hydroxyethyl starch, polydextrose, gum arabic, and pectin, and an electrolyte (Japanese Unexamined Patent Application, First Publication No. H2-25424, Japanese Unexamined Patent Application, First Publication No. H3-206046), a preparation which is obtained by combining erythritol or xylitol and an electrolyte (Japanese Unexamined Patent Application, First Publication No. H3-284620), a preparation which is obtained by combining a fructooligo saccharide and an electrolyte (Japanese Unexamined Patent Application, First Publication No. H3-291228), a composition containing at least one selected from lactitol, maltitol, and carboxymethyl cellulose, and an electrolyte (Japanese Unexamined Patent Application, First Publication No. H5-255092), magnesium citrate (Japanese Unexamined Patent Application, First Publication No. H5-306221), and an isotonic aqueous solution obtained by using a saline purgative agent such as sodium dihydrogen phosphate or disodium hydrogen phosphate, and a tonicity adjusting agent (for example, a purgative agent for an large intestine examination or an abdominal surgery pretreatment having magnesium citrate as a saline purgative agent, “Magcorol (registered trademark) P” (manufactured by Horii Pharmaceutical Ind., Ltd.)).

The concentration of the barium sulfate in the liquid preparation for oral administration A according to the present invention is preferably 0.15 g/mL to 0.35 g/mL, more preferably 0.15 g/mL to 0.30 g/mL, still more preferably 0.15 g/mL to 0.28 g/mL, and particularly preferably 0.15 g/mL to 0.25 g/mL. The concentration range is lower than that in a barium formulation in the related art. Since the total amount of barium sulfate required for labeling residues or remaining water in the intestinal tract is substantially determined, in general, as the concentration of the barium sulfate is increased, the liquid volume to be taken in order to label the residues or the remaining water in the intestinal tract is decreased, and as the concentration of the barium sulfate is decreased, the liquid volume to be administered is increased.

The liquid preparation for oral administration A according to the present invention has flow characteristics in which the viscosity at a shear rate of 21.54 s⁻¹ is 100 mPa·s or greater, and the viscosity at a shear rate of 464.1 s⁻¹ is 5 mPa·s to 90 mPa·s. That is, the liquid preparation for oral administration A has higher fluidity than that in barium formulations in the related art in a state in which the shear rate is high, and has flow characteristics such that the viscosity is sufficiently high in a state in which the shear rate is low. The viscosity of the liquid preparation for oral administration A at a shear rate of 21.54 s⁻¹ is preferably 150 mPa·s or greater, more preferably 250 mPa·s or greater, still more preferably 350 mPa·s or greater, still more preferably 350 mPa·s to 750 mPa·s, and particularly preferably 350 mPa·s to 500 mPa·s. The viscosity of the liquid preparation for oral administration A at a shear rate of 464.1 s⁻¹ is preferably 10 mPa·s to 70 mPa·s, more preferably 10 mPa·s to 60 mPa·s, and still more preferably 20 mPa·s to 60 mPa·s.

The viscosity of the liquid preparation for oral administration A can be adjusted by suitably adjusting the concentration of barium sulfate to be contained, the composition of the aqueous medium in which the barium sulfate is dispersed, the type or the amount of thickening agent to be used, or the like. Moreover, in the present invention and the present specification, the viscosity of a liquid preparation containing barium sulfate at each shear rate is a value measured while continuously changing the shear rate within a range of 0.01 s⁻¹ to 1000 s⁻¹ using a rotational viscometer. For example, the viscosity of a sample (1.2 mL) is measured while the shear rate is continuously changed within a range of 0.01 s⁻¹ to 1000 s⁻¹ at 25° C. using a cone-plate type rotational viscometer “MCR300” (manufactured by Anton Paar GmbH) and a cone plate “CP50-2”.

In the liquid preparation for oral administration A according to the present invention, components other than the barium sulfate, the thickening agent, and the aqueous medium can be contained within a range not interfering with the effects of the present invention. As the other components, components the same as in a liquid preparation for oral administration B described below are exemplary examples.

The liquid preparation for oral administration A according to the present invention is preferably used in a pretreatment for digestive tract imaging by the CT colonography. The liquid preparation for oral administration A can be used in the same manner as in other contrast agents used in a pretreatment for the CT colonography. For example, 10 mL to 100 mL of the liquid preparation for oral administration A may be taken after cleaning the intestinal tract by taking about 1 L to 2 L of the oral intestinal tract cleaning agent on the examination day of the CT colonography, or by taking about 1 L to 2 L of the liquid preparation for oral administration A prepared by mixing the barium sulfate or the like in the oral intestinal tract cleaning agent, intestinal tract cleaning and residue labeling may be performed at the same time. In addition, during the period from after the dinner on the day before examination by the CT colonography is performed to bedtime, 10 mL to 100 mL of the liquid preparation for oral administration A may be taken, or the liquid preparation for oral administration A may be taken in the same administration mode as in the liquid preparation for oral administration B described below. That is, 50 mL to 2000 mL (preferably 50 mL to 1200 mL, and more preferably 70 mL to 900 mL) of the liquid preparation for oral administration A prepared by mixing the barium sulfate or the like in the oral intestinal tract cleaning agent may be taken all at once or in several parts over a period of time on the day before examination by the CT colonography is performed.

[Liquid Preparation for Oral Administration B]

The liquid preparation for oral administration according to the second aspect of the present invention (hereinafter, also referred to as “liquid preparation for oral administration B”) is a liquid preparation for oral administration used in digestive tract imaging by the CT colonography and contains barium and a water-soluble polymer or a saline purgative agent, and 50 mL to 2000 mL, preferably 50 mL to 1200 mL, more preferably 70 mL to 900 mL, still more preferably 100 mL to 900 mL of the liquid preparation for oral administration B is taken all at once or in several parts over a period of time on the day before the CT colonography is performed.

As the mode of taking the liquid preparation for oral administration B according to the present invention, on the day before the CT colonography is performed, 50 mL to 2000 mL (preferably 50 mL to 1200 mL, and more preferably 70 mL to 900 mL) of the liquid preparation for oral administration B may be taken, 50 mL to 2000 mL may be taken in a single dose, or may be taken in several divided doses. For example, 100 mL to 900 mL may be taken two or three times by dividing, or 100 mL to 400 mL can also be taken in a single dose.

In addition, a single dose may be taken all at once, or over a period of 30 minutes. Moreover, the amount of the liquid preparation for oral administration B according to the present invention taken on the day before the CT colonography is performed is within a range of 50 mL to 2000 mL (preferably 50 mL to 1200 mL, and more preferably 70 mL to 900 mL), and can be determined according to the age, the weight, or the condition of a subject of the CT colonography. In addition, the liquid preparation for oral administration may be taken after meals, may be taken before meals or at the same time as meals, or may be taken after waking up or between meals, or before falling asleep. In a case where the total dose is a lower volume, it is preferable to take the liquid preparation for oral administration B in two or three installments rather than all at once. In a case where the dose is a low volume depending on the state of bowel movements of a recipient, there is a case where a relatively large amount of solid residues remains in the intestinal tract; however, when a sufficient amount of barium is contained in the liquid preparation for oral administration B according to the present invention, it is possible to sufficiently label the solid residues in the intestinal tract, and it is possible to use the captured image in diagnosis.

Moreover, at the time of dosing the liquid preparation for oral administration B according to the present invention, it is preferable to sufficiently take in water. For example, on the day before the CT colonography is performed, after taking the liquid preparation for oral administration B according to the present invention, it is preferable to take in about 50 mL to 300 mL of water.

<Barium>

In the liquid preparation for oral administration B according to the present invention, barium is used as a contrast agent. Since the barium has a high CT value, it is possible to sufficiently tag the entire large intestine at a relatively small dose. The barium used in the liquid preparation for oral administration B according to the present invention is the barium generally used in the large intestine examination or the like by the CT colonography or barium enema examination such as barium sulfate. Specific examples of the barium include barium sulfate formulations such as Bamstar S200 (manufactured by Kyosei Pharmaceutical Co., Ltd.) and Barytgen (registered trademark) CT (manufactured by Fushimi pharmaceutical Co., Ltd.).

When the residues or the remaining stool in the intestinal tract are labeled with barium sulfate, performing uniform labeling is one of the conditions that lead to a good image. Therefore, in the liquid preparation for oral administration B according to the present invention, barium having a specific particle size distribution (specifically, a particle size distribution in which the particle diameter D10 is 0.3 μm to 0.5 μm and the particle diameter D90 which is a diameter when the accumulation value becomes 90% is 1.0 μm to 2.0 μm) used in the liquid preparation for oral administration A is preferably used. In this case, the barium is particularly preferably contained so as to have the same flow characteristics or the same concentration of barium as in the liquid preparation for oral administration A. By using the same type of barium as in the liquid preparation for oral administration A, in a case where the liquid preparation for oral administration B is taken, it is possible to further efficiently perform mixing of the barium and the object to be labeled (residues) due to the motion of the stomach and intestines, and precipitation of barium sulfate in the remaining water in the intestinal tract is suppressed, and due to this, it is possible to further uniformly label the entire large intestine.

The CT value of a liquid residue-storing portion can be adjusted depending on the barium content or the dose of the liquid preparation for oral administration B according to the present invention taken on the day before the CT colonography is performed. Therefore, the barium content of the liquid preparation for oral administration B according to the present invention can be suitably adjusted in consideration of the dose, the CT value of the residues in the CT colonography after taking, the condition of a subject, and the like. For example, the barium content of the liquid preparation for oral administration B according to the present invention can be determined while considering the taking mode such that the CT value of the residues becomes 300 HU or greater, and preferably, the CT value is uniformly maintained within a range of 300 HU to 700 HU at each portion in the digestive tract, in the CT colonography in the case of performing a pretreatment using the liquid preparation for oral administration. Specifically, the barium content of the liquid preparation for oral administration B according to the present invention is preferably 8.3 mg/mL to 2000 mg/mL, as the concentration of the barium sulfate. Actually, there is a report that, in humans, in a case where 750 mL of 2.1 w/v % (0.021 g/mL) barium sulfate suspension is administered in three parts over the course of a day before the CT colonography is performed, or similarly, in a case where 50 mL of 40 w/v % (0.4 g/mL) barium sulfate suspension is taken in three parts over the course of a day before the CT colonography is performed, the CT colonography is possible (American Journal of Roentgenology, Vol. 183, no. 4, p 945-948). The total dose of barium taken in by taking the liquid preparation for oral administration B according to the present invention on the day before the CT colonography is performed is preferably 6 g to 600 g, and more preferably 10 g to 100 g as the barium sulfate.

In particular, the liquid preparation for oral administration B according to the present invention contains barium sulfate which has the particle diameter D10 of 0.3 μm to 0.5 μm and the particle diameter D90 which is a diameter when the accumulation value becomes 90% of 1.0 μm to 2.0 μm, and preferably has the viscosity of 350 mPa·s or greater at a shear rate of 21.54 s⁻¹, and 5 mPa·s to 90 mPa·s at a shear rate of 464.1 s⁻¹. The concentration of the barium sulfate in the liquid preparation for oral administration B in this case is preferably 0.15 g/mL to 0.35 g/mL, more preferably 0.15 g/mL to 0.30 g/mL, still more preferably 0.15 g/mL to 0.28 g/mL, and particularly preferably 0.15 g/mL to 0.25 g/mL.

The liquid preparation for oral administration B according to the present invention includes al least one of a water-soluble polymer or a saline purgative agent, in addition to barium. In the present invention, in order to further reduce the burden on a subject, barium and the water-soluble polymer is preferably contained. Moreover, the liquid preparation for oral administration B according to the present invention may include both the water-soluble polymer and the saline purgative agent.

<Water-Soluble Polymer>

Examples of the water-soluble polymer contained in the liquid preparation for oral administration B according to the present invention can include polyethylene glycol, polydextrose, dextran, dextrin, hydroxyethyl starch, gum arabic, pullulan, pectin, carboxymethyl cellulose, carboxymethyl cellulose salts such as carboxymethyl cellulose sodium, carrageenan, xanthan gum, gum tragacanth, croscarmellose sodium, and crystalline cellulose-carmellose sodium. Among these, a water-soluble polymer selected from the group consisting of polyethylene glycol, polydextrose, dextran, hydroxyethyl starch, gum arabic, pullulan, and pectin is preferable from the view point of formulation stability, and polyethylene glycol is particularly preferably contained. The molecular weight of polyethylene glycol is preferably in a range of 2000 to 8000, and more preferably in a range of 3000 to 7000.

<Saline Purgative Agent>

Examples of the saline purgative agent contained in the liquid preparation for oral administration B according to the present invention can include magnesium citrate, sodium dihydrogen phosphate, and disodium hydrogen phosphate. The concentration of the saline purgative agent is not particularly limited as long as it is a concentration at which an laxative action can be exhibited, and it is possible to suitably adjust the concentration in consideration of the type of the saline purgative agent, the condition of a subject who takes the saline purgative agent, and the like. For example, in a case where the concentration of sodium ion is 60 mEq/L, the concentration of potassium ion is 20 mEq/L, the concentration of magnesium ion is 3 mEq/L, the concentration of chloride ion is 50 mEq/L, the concentration of phosphate is 10 mmol/L, the concentration of citrate ions 35 mEq/L, and the concentration of lactate ion is 20 mEq/L, the balance of the electrolytes in the living body is not excessively affected thereby. Therefore, the saline purgative agent is added such that the amount thereof in the liquid preparation for oral administration B according to the present invention becomes higher than the concentrations above.

<Osmotic Pressure>

The osmotic pressure of the liquid preparation for oral administration B according to the present invention is preferably 200 mOsm/L to 570 mOsm/L, more preferably 200 mOsm/L to 440 mOsm/L, and still more preferably 250 mOsm/L to 320 mOsm/L. When the osmotic pressure is within the above-described range, it is possible to limit variations in the serum electrolyte balance and the osmotic pressure in the living body due to taking the liquid preparation for oral administration to be a minimum. The liquid preparation for oral administration B according to the present invention is more preferably adjusted to be within an osmotic pressure range of isotonicity of 280 mOsm/L to 320 mOsm/L or an osmotic pressure range close to isotonicity.

The liquid preparation for oral administration B according to the present invention may be a formulation which adjusts the osmotic pressure in the intestinal tract in combination with taking of water or the like, for example, as the intestinal tract cleaning liquid described in Japanese Patent No. 4131266. In this manner, in a case of being taken in combination with water or the like, for example, the osmotic pressure of the liquid preparation for oral administration B according to the present invention may be a higher osmotic pressure than the above-described osmotic pressure range, for example, 900 mOsm/L or less, preferably 850 mOsm/L or less, and more preferably 700 mOsm/L or less. That is, the osmotic pressure of the liquid preparation for oral administration B according to the present invention is preferably 200 mOsm/L to 900 mOsm/L.

The liquid preparation for oral administration B according to the present invention preferably includes an electrolyte in order to adjust the osmotic pressure to be a desired value. The type and the mixing amount of electrolyte contained in the liquid preparation for oral administration according to the present invention is suitably selected such that the osmotic pressure of the liquid preparation for oral administration is within the above-described range.

The electrolyte refers to a substance which becomes ions by dissociation in a solution, and examples thereof include Na⁺, K⁺, Ca²⁺, Mg²⁺, Cl⁻, HCO₃⁻, so₄²⁻, HPO₄²⁻, an organic acid group, and an organic base. For example, the saline purgative agent is also included in the electrolyte. Examples of the electrolyte contained in the liquid preparation for oral administration B according to the present invention can include the same type of electrolyte as used in an electrolyte infusion or the like to be intravenously administered. More specifically, as the sodium ion source, sodium chloride, sodium acetate, sodium citrate, sodium dihydrogen phosphate, disodium hydrogen phosphate, sodium sulfate, and sodium lactate can be exemplified, as the potassium ion source, potassium chloride, potassium acetate, potassium citrate, potassium dihydrogen phosphate, dipotassium hydrogen phosphate, potassium sulfate, and potassium lactate can be exemplified, as the calcium ion source, calcium chloride, calcium gluconate, calcium pantothenate, calcium lactate, calcium acetate, and calcium glycerophosphate can be exemplified, as the magnesium ion source, magnesium sulfate, magnesium chloride, magnesium acetate, and magnesium citrate can be exemplified, as the phosphate ion source, sodium dihydrogen phosphate, disodium hydrogen phosphate, and sodium glycerophosphate can be exemplified, as the chlorine ion source, sodium chloride, potassium chloride, calcium chloride, and magnesium chloride are exemplary examples, and as the bicarbonate ion source, sodium hydrogen carbonate is an exemplary example, respectively, and these compounds may be in the hydrate forms.

<Thickening Agent>

In a case where barium sulfate is contained as barium, the liquid preparation for oral administration B according to the present invention preferably further contains a thickening agent. By increasing viscosity, it is possible to prevent the barium sulfate from being precipitated in the intestinal tract. The thickening agent added to the liquid preparation for oral administration B according to the present invention is not particularly limited as long as it can be orally taken in and it does not cause a particular reaction with various components such as barium sulfate, and it can be suitably selected from among known thickening agent and used. Specific examples of the thickening agent can include guar gum, carrageenan, xanthan gum, fenugreek gum, pectin, pullulan, gum arabic, dextran, dextrin, polydextrose, hydroxyethyl starch, carboxymethyl cellulose, carboxymethyl cellulose salts such as carboxymethyl cellulose sodium (CMC-Na), hydroxypropyl cellulose, gum tragacanth, sodium alginate, sodium chondroitin sulfate, locust bean gum, gellan gum, croscarmellose sodium, and crystalline cellulose-carmellose sodium.

<Saccharide>

The liquid preparation for oral administration B according to the present invention may further include a saccharide. In particular, as the liquid preparation for oral administration B preferable to a pretreatment for CT colonography which is performed as a screening test, since formulation stability, patient acceptability including easiness of taking, or the like is excellent, it is also preferable to add a mixture of an electrolyte and a saccharide.

The saccharide contained in the liquid preparation for oral administration B according to the present invention may be any one of a saccharide and a sugar alcohol. Specific examples of saccharide contained in the liquid preparation for oral administration B according to the present invention can include sucrose, sorbitol, xylitol, erythritol, mannitol, trehalose, lactitol, lactulose, maltitol, palatinose, raffinose, and glycerin. Among these, xylitol, sorbitol, lactulose, lactitol, and raffinose are particularly preferable from the viewpoint of the formulation stability, the patient acceptability including easiness of dosing.

<Mixing>

In the liquid preparation for oral administration B according to the present invention, the sum of the mixing amount of a water-soluble polymer and an electrolyte or the sum total of the mixing amount of a water-soluble polymer, an electrolyte, and a saccharide is 2 g to 40 g, and preferably 10 g to 30 g in 400 mL of a liquid formulation, for example. In order to maintain the serum electrolyte balance by canceling the amount of electrolyte which is absorbed by the intestinal tract and the amount of electrolyte which is secreted from the intestinal tract, the electrolyte is preferably added such that the liquid preparation for oral administration B according to the present invention becomes Na⁺: 30 mEq/L to 150 mEq/L, K⁺: 3 mEq/L to 20 mEq/L, Cl⁻: 20 mEq/L to 70 mEq/L, and HCO₃⁻: 10 mEq/L to 50 mEq/L. In order to limit the absorption of water and electrolytes in the intestinal tract, in addition to these electrolytes, poorly absorbed ions such as magnesium ions and sulfate ions are preferably added in the liquid preparation for oral administration B. In the case of magnesium ions, the magnesium ions are preferably mixed in while adjusting such that the concentration of the magnesium ions in the liquid preparation for oral administration B becomes 40 mEq/L to 120 mEq/L. In the case of sulfate ions, the sulfate ions are preferably mixed in while adjusting such that the concentration of the sulfate ions in the liquid preparation for oral administration B becomes 40 mEq/L to 120 mEq/L. In the case of using a saccharide in addition to the electrolyte and the water-soluble polymer, the amount of saccharide used is 2 g to 40 g, and preferably 5 g to 10 g in 400 mL of the liquid preparation for oral administration B.

<Digestive Tract Function-Promoting Agent>

The liquid preparation for oral administration B according to the present invention preferably further contains a digestive tract function-promoting agent, in addition to the barium and the water-soluble polymer (or saline purgative agent). By using the digestive tract function-promoting agent, the liquid preparation for oral administration which is taken or the mixture of the liquid preparation for oral administration and the intestinal tract content is likely to be excreted, and it is possible to reduce the amount of the residues in the intestinal tract at the time of the CT colonography.

Examples of the digestive tract function-promoting agent contained in the liquid preparation for oral administration B according to the present invention include a serotonin 5-HT₄ receptor stimulator. Examples of the serotonin 5-HT₄ receptor stimulator can include a benzamide derivative, cisapride, and metoclopramide represented by the general formula described in Claim 1 of Japanese Examined Patent Application, Second Publication No. H3-54937 including mosapride citrate. Among these compounds, the mosapride citrate is most preferable (Medical Review Co., Ltd., published in 1998, “Mosapride and Gastrointestinal motility”) since side effects such as arrhythmia induction are low. The mosapride citrate is preferably contained in the liquid preparation for oral administration B according to the present invention while suitably adjusting such that the daily dose (that is, the amount used in the pretreatment for a single CT colonography) becomes 5 mg to 40 mg.

A large intestine stimulant laxative which is widely used can also be contained in the liquid preparation for oral administration B according to the present invention as a digestive tract function-promoting agent. Examples of the large intestine stimulant laxative include sodium picosulfate, bisacodyl, and sennoside. The large intestine stimulant laxative can be contained in the liquid preparation for oral administration B according to the present invention while suitably adjusting such that the amount used in the pretreatment for a single CT colonography becomes the daily dose of the formulation regulated or the amount regulated as the dose for the pretreatment for the large intestine examination. For example, the sodium picosulfate is preferably contained in the liquid preparation for oral administration B according to the present invention while suitably adjusting such that the daily dose (that is, the amount used in the pretreatment for a single CT colonography) becomes 5 mg to 150 mg.

<Dimethyl Polysiloxane>

The liquid preparation for oral administration B according to the present invention preferably further contains dimethyl polysiloxane, in addition to the barium and the water-soluble polymer (or saline purgative agent). Since the dimethyl polysiloxane exhibits defoaming action with respect to foam in a foaming viscous liquid, it is possible to improve the reading accuracy and the reading efficiency of the lesion from a captured image. Examples of the dimethyl polysiloxane contained in the liquid preparation for oral administration B according to the present invention include a commercially available “GASCON (registered trademark)” as a gas eliminator in the digestive tract. The amount of dimethyl polysiloxane contained in the liquid preparation for oral administration B according to the present invention is preferably contained in the liquid preparation for oral administration B according to the present invention while suitably adjusting such that the daily dose (that is, the amount used in the pretreatment for a single CT colonography) of an adult becomes 30 mg to 120 mg, and preferably 40 mg to 80 mg. Moreover, on the day before the CT colonography is performed, the liquid preparation for oral administration B according to the present invention not containing dimethyl polysiloxane and a liquid preparation including dimethyl polysiloxane each may be independently taken orally all at once or in several parts over a period of time.

<Sweetener•Flavoring>

Barium, the water-soluble polymer, and the electrolyte have a specific taste and smell in many cases. Accordingly, taste or smell correcting means is preferably applied to the liquid preparation for oral administration B according to the present invention. Specifically, it is possible to add a sweetener or a flavoring thereto.

The flavoring contained in the liquid preparation for oral administration B according to the present invention is not particularly limited as long as it masks the odor resulting from the water-soluble polymer or the electrolyte when orally taking in the liquid preparation for oral administration. In the present invention, food flavorings are suitable, and, in particular, fruit flavorings are suitable. Examples of the fruit flavorings include flavorings of lemon, orange, grapefruit, lemon lime, mandarin orange, grape, strawberry, cherry, apple, apricot, and raspberry. Among these, in order to give a fresh drinking feeling, liquid flavorings of citrus such as lemon, orange, grapefruit, and lemon lime are optimal. As the liquid flavorings, it is possible to use essential oils obtained from these fruits by a squeeze method or a steam distillation. In addition, limonene, citral, citronellal, linalool, and octanal can be used alone or a mixture thereof can be used. Among these, the flavoring of citrus including limonene is preferably used. Such flavorings are commercially available from perfume manufacturers, and it is practical to use these. Moreover, in the liquid flavorings, a water-soluble liquid flavoring obtained by extracting or dissolving the flavoring components with a water-containing alcohol or the like, or a oil-soluble liquid flavoring obtained by dissolving the flavoring components in an oily solvent is included. A flavoring obtained by adding a preservative or the like into the flavoring components (essential oil) is also included therein. As the liquid flavoring, the use of an oil-soluble flavoring or a flavoring derived from an essential oil is more preferable than a water-soluble liquid flavoring from the viewpoint of storage stability.

The sweetener used in the present invention is intended to be easy to drink when taking the liquid preparation for oral administration. The sweetener must be a sweetener which does not generate a hydrogen gas or a methane gas, or generates an extremely small amount of hydrogen gas or methane gas in the intestine. However, in a case where an endoscopic examination is performed after capturing by the CT colonography, and a technique (endoscopic polypectomy) of electrically excising a polyp at the same time as the endoscopic examination is not used, the feeling of administration of a liquid preparation and energy supply may be improved by actively adding a saccharide, and in particular, sucrose (purified sucrose). As a sweetener which does not generate a hydrogen gas and a methane gas or generates extremely small amount of hydrogen gas or methane gas in the intestine, specifically, saccharin, sodium saccharin, acesulfame-K, cyclamate (sodium cyclohexylsulfamate), and aspartame are preferably used singly or in combination. The preferred added amount of these sweeteners is 0.001% by mass to 3% by mass, and more preferably 0.01% by mass to 0.3% by mass of the liquid preparation for oral administration.

<Other Components>

In the liquid preparation for oral administration B according to the present invention, other components can be contained within a range not interfering with the effects of the present invention. For example, in the liquid preparation for oral administration B according to the present invention, ascorbic acid and/or one or more salts (ascorbate component) thereof may be contained. For example, the ascorbate component is preferably added within a range of 4 g to 15 g per 1 L of the liquid preparation for oral administration B according to the present invention. In particular, in a case where the total amount to be taken on the day before the CT colonography is performed is a relatively low volume, the liquid preparation for oral administration B according to the present invention preferably further contains the ascorbate component.

Preferred salts of ascorbic acid are alkali metal salts and alkaline earth metal salts, such as sodium ascorbate, potassium ascorbate, magnesium ascorbate, and calcium ascorbate. A particularly preferred salt of ascorbic acid is sodium ascorbate. Preferably, the ascorbate component includes both ascorbic acid and one or more salts thereof. Preferably, ascorbic acid and a salt thereof is present in a weight ratio in a range of 1:9 to 9:1. Ascorbic acid and salt thereof, actually, can be provided as hydrates. If a hydrate is used, the weight and/or weight ratio mentioned here is the weight and/or weight ratio of ascorbic acid or a salt thereof excluding the hydrated water. Preferably, ascorbic acid and a salt thereof are present within a range of 2:8 to 8:2, more preferably 3:7 to 7:3, and still more preferably 4:6 to 6:4, and, for example, are present in a weight ratio of 4.7:5.9.

<Oral Intestinal Tract Cleaning Agent and Contrast Agent>

The liquid preparation for oral administration B according to the present invention may be a liquid preparation for oral administration obtained by adding a contrast agent having barium as an effective component to a liquid oral intestinal tract cleaning agent (intestinal tract cleaning liquid) used when performing intestinal tract cleaning as a pretreatment for endoscopic examination of large intestine, barium enema examination, or the CT colonography. That is, a mixture obtained by mixing a barium contrast agent in the oral intestinal tract cleaning agent can be used as the liquid preparation for oral administration B according to the present invention. As such an oral intestinal tract cleaning agent, for example, those exemplary examples of the liquid preparation for oral administration A described above can be used.

<Container>

The liquid preparation for oral administration B according to the present invention may be a form of filling a metal container such as an aluminum can, a glass container, a flexible container such as a plastic container, or a rigid or semi-rigid container in the same manner as other liquid preparations. Examples of the plastic container include a container formed of a polyolefin-based thermoplastic resin film. Examples of the rigid or semi-rigid container include a PET bottle manufactured using polyethylene terephthalate as a raw material.

In particular, in a case where the liquid preparation for oral administration B according to the present invention contains sodium hydrogen carbonate as a component, there is a concern that the sodium hydrogen carbonate reacts with the moisture in the air during storage, and due to this, carbon dioxide is generated. Therefore, in this case, the liquid preparation for oral administration B according to the present invention preferably fills a container having low gas permeability (preferably, 20 cc/m²·day·atm (25° C.) or less), that is, a metal can such as an aluminum can, a glass container, or a bag-shape container made of a metal such as aluminum, which is lined with a thermoplastic resin. In the case of filling a container having high gas permeability, the container may be further packaged externally with a package having low gas permeability.

Furthermore, in a case where the liquid preparation for oral administration B according to the present invention contains sodium hydrogen carbonate as a component, the inside of the container is preferably filled with a gas having a high carbon dioxide gas partial pressure in order to suppress the variation over time in the concentration of sodium hydrogen carbonate.

<Preparation at Time of Use>

The liquid preparation for oral administration according to the present invention can be prepared by preparing a concentrated liquid containing respective components in advance and diluting the concentrated liquid with the appropriate amount of water at the time of dosing. The concentrated liquid containing respective components may be a form of filling an aluminum can, a flexible container, or a rigid or semi-rigid container. At this time, the concentrate liquid is, of course, sterilized or pasteurized by a common method (Japanese Unexamined Patent Application, First Publication No. H9-58747, Japanese Unexamined Patent Application, First Publication No. H8-253220, or the like).

In addition, by preparing respective components which are powdered, tableted, or granulated, and by dissolving or suspending these in the appropriate amount of water at the time of dosing, the liquid preparation for oral administration according to the present invention can also be prepared. After preparing a composition obtained by mixing two or more types of components in advance, at the time of dosing, the composition and other components may be dissolved or suspended in the appropriate amount of water.

For example, the liquid preparation for oral administration B according to the present invention can be prepared by dissolving and suspending both a powder formulation with which an intestinal tract cleaning liquid which contains any one of a water-soluble polymer and a saline purgative agent, and if necessary, an electrolyte other than the saline purgative agent, and has an osmotic pressure of 200 mOsm/L to 900 mOsm/L (preferably 200 mOsm/L to 570 mOsm/L, and more preferably 200 mOsm/L to 440 mOsm/L) by being dissolved in a predetermined amount of water can be prepared (for example, “Niflec” (registered trademark) mixed internal drug (manufactured by Ajinomoto Pharmaceuticals Co., Ltd.) according to the present invention described in Japanese Unexamined Patent Application, First Publication No. H1-125319 described above) and a contrast agent having barium as an effective component in a predetermined amount of water at the time of dosing. Moreover, instead of the powder formulation, formulations formulated in form of a tablet or a granule can be used.

In addition, after preparing a composition containing at least any one of a water-soluble polymer and a saline purgative agent, and barium (if necessary, an electrolyte other than the saline purgative agent, dimethyl polysiloxane, and a digestive tract function-promoting agent) in advance, at the time of dosing, the composition and other components such as a sweetener and a flavoring may be dissolved in the appropriate amount of water. By preparing the effective components of the oral intestinal tract cleaning agent or the contrast agent as one composition in advance, and by making the components having taste or smell correcting effect such as a sweetener or a flavoring be individually adjustable, it is possible to further conveniently prepare the liquid preparation for oral administration B having the desired taste and smell.

[Composition for Imaging Digestive Tract]

A composition including at least barium and a water-soluble polymer or a saline purgative agent among the components constituting the liquid preparation for oral administration B according to the third aspect of the present invention is referred to as the composition for imaging a digestive tract according to the present invention. That is, the composition for imaging a digestive tract according to the present invention contains barium and at least any one of a water-soluble polymer and a saline purgative agent, and 50 mL to 2000 mL (preferably, 50 mL to 1200 mL, and more preferably 70 mL to 900 mL) of the solution obtained by suspending or diluting in water is taken all at once or in several parts over a period of time on the day before the CT colonography is performed. Moreover, a composition including components such as an electrolyte other than the saline purgative agent, dimethyl polysiloxane, the digestive tract function-promoting agent, a sweetener, and a flavoring is also included in the composition for imaging a digestive tract according to the present invention. In addition, the osmotic pressure of the aqueous solution obtained by dissolving or diluting the composition for imaging a digestive tract according to the present invention in water is preferably 200 mOsm/L to 900 mOsm/L, more preferably 200 mOsm/L to 570 mOsm/L, and still more preferably 200 mOsm/L to 440 mOsm/L. The viscosity of the aqueous solution obtained by dissolving or diluting the composition for imaging a digestive tract according to the present invention in water preferably is 350 mPa·s or greater at a shear rate of 21.54 s⁻¹, and preferably 5 mPa·s to 90 mPa·s at a shear rate of 464.1 s⁻¹.

The composition for imaging a digestive tract according to the present invention is preferably a composition obtained by mixing components excluding barium, constituting the liquid preparation for oral administration B according to the present invention after adjusting the particle diameter such that respective components becomes mixable.

In order to achieve easiness of taking and convenience of use, a composition containing all the components constituting the liquid preparation for oral administration B according to the present invention including barium is preferably adjusted such that a single dose taken all at once or in several parts over a period of time becomes the amount per packaging material.

<Package>

The composition for imaging a digestive tract according to the present invention fills a package such as a bag or a can made of metals such as aluminum, which is lined with a thermoplastic resin, or a plastic package formed of a thermoplastic resin. In a case where a component which may be adsorbed onto the package or a component which may be transmitted and dissipated from the package of a gas or a volatile component is included in the composition for imaging a digestive tract according to the present invention, the thickness of the resin layer also influences the adsorption amount or the transmitted and dissipated amount, along with the area of the inside of the package, and thus, reduction in sealing strength of the package does not occur, and it is necessary to set the thickness of the resin layer within a range in which a sufficient amount of components remains during use.

In particular, in a case where sodium hydrogen carbonate is included as an effective component, carbon dioxide gas is generated over time by a reaction with the moisture in the air. Accordingly, in order to prevent carbon dioxide from dissipating from the package and the sodium hydrogen carbonate content from being reduced, the gas permeability of the package is preferably 20 cc/m²·day·atm (25° C.) or less.

The composition for imaging a digestive tract according to the present invention can be taken after being transferred from the package to a container and being suspended in water. However, in the case of using the package itself as a plastic container in which required water for suspending (for example, 50 mL to 2000 mL, preferably 50 mL to 1200 mL, and more preferably 70 mL to 900 mL) can be stored, and taking after putting water directly into the package during use and suspending, it is convenient because the need to separately prepare a container for suspending is eliminated. In addition, it is preferable to make the scale by which the dose per one time to be taken by dividing split can be seen, on the package.

As the plastic container in this case, a container formed of a polyolefin-based thermoplastic resin is preferable. Examples of the polyolefin-based thermoplastic resin can include polyethylene and polypropylene. Although soft polyolefin has favorable adhesion between resins necessary when molding the container, the soft polyolefin has high gas adsorption/permeability. On the other hand, although rigid polyolefin has low gas adsorption/permeability, adhesion between resins is poor. Therefore, if using the soft polyolefin on the inner layer (surface in contact with the composition for imaging a digestive tract) and overlaying rigid polyolefin on the soft polyolefin in order to satisfy both of the requirements in molding and the requirements of gas adsorption/permeability requirements, it is possible to obtain a large volume of a package which overall has low adsorption/permeability, favorable adhesion (formability), and can also serve as a container for suspending. Furthermore, the package particularly preferably has the total three layers in which the soft polyolefin is used on the innermost layer (surface in contact with the composition for imaging a digestive tract), the rigid polyolefin is overlaid on the outside thereof, and the soft polyolefin is further overlaid on the outside of the rigid polyolefin.

It is particularly preferable to use the soft polyethylene, in particular, a linear polyethylene on the innermost layer. The thickness of the innermost layer in this case is 100 μm or less, and preferably 50 μm, the layer is preferably formed of a polyolefin-based thermoplastic resin in order to suppress the reduction of various components due to adsorption or transmission and dissipation. In the case of forming a plastic package with two layers, that is, the inner layer formed of the soft polyolefin and the outer layer formed of the rigid polyolefin, it is preferable that the total thickness is 70 μm to 200 μm, and each thickness of the inner layer and the outer layer is 35 μm to 100 μm. In the case of forming a plastic package formed of three layers by overlaying the soft polyolefin on the innermost layer, the rigid polyolefin on the outside thereof, and the soft polyolefin on the outside of the rigid polyolefin, it is preferable that the total thickness is 70 μm to 200 μm, the thickness of each layer is 15 μm to 70 μm and, in particular, is 20 μm to 40 μm.

It is preferable to form a package with enhanced strength against drop impact of or piercing through the package by laminating a thermoplastic resin such as polyethylene terephthalate or polyamide on the further outside of the polyethylene layer of the two layers or three layers.

Although the shape of the package is not particularly limited, in a case where the package is also used as the plastic container which can store the water for suspending, the package is preferably equipped with an inlet of the water for suspending, and is preferably provided with a bottom by which the package can stand when suspended. In consideration of manufacturing convenience, the package preferably has two side walls having a substantially triangular shape with vertically longer height than the base, an inlet attached on the top portion of the side wall, and the bottom having a shape similar to a camellia leaf attached on the bottom portion of the side wall. The two side walls and the bottom are constituted with flexible plastic, the inlet is constituted with the rigid plastic, and a lid is attached to the inlet. By constituting the two side walls and the bottom with flexible plastic and making the bottom have an internal collapsible structure until the water for suspending is put, in the case of eliminating the air from the package (container) until injecting the water for suspending, the package can become compact, and thus, storage and transportation are convenient. Two side walls are preferably constituted with a transparent or translucent plastic so that the amount of the contents can be confirmed by eye. By making the scale indicating the amount of water for suspending on the side wall, when taking, the dose can be confirmed by eye without requiring a meter when injecting the water for suspending.

The composition for imaging a digestive tract according to the present invention can be prepared by mixing the predetermined components by a method known in the related art. In a case where the package is filled, air is eliminated as much as possible, and due to this, it is possible to prevent quality degradation over time of the effective components unstable with respect to oxygen, and since the package is not bulky, storage and transportation are convenient. In addition, in a case where long term storage is required, after putting the package into an exterior bag having gas impermeability formed of aluminum laminate film or the like, the bag can be sealed.

<Kit or Packaged Goods>

Respective components and compositions used for preparing the liquid preparation for oral administration B according to the present invention including the composition for imaging a digestive tract according to the fourth aspect of the present invention is preferably made as a kit or packaged goods. For example, it is possible to use one obtained by combining the oral intestinal tract cleaning agent and the barium contrast agent described above, one obtained by combining separately prepared barium and the water-soluble polymer so as to be mixable, one obtained by combining separately prepared barium and the saline purgative agent so as to be mixable, or one obtained by combining separately prepared barium, the water-soluble polymer, and the saline purgative agent so as to be mixable as the drug for pretreatment for the CT colonography.

Such kits and packaged goods are not particularly limited on the fabrication thereof, and the fabrication can be suitably performed according to a common method. That is, in one packaging form, at least each component and composition used for preparing the liquid preparation for oral administration B according to the present invention may be included. In these kits and packaged goods, an instruction in which the taking order and the taking method are described, and a note in which symptoms in which taking the drug must be stopped and countermeasure at the time of occurrence of side effects are described are preferably provided. Furthermore, a cup, a check sheet of dose, a watch, a liquid for suspending, and the like can be provided.

As an example, a plastic container is filled with a powder formulation with which an intestinal tract cleaning liquid which contains an electrolyte and a water-soluble polymer (if necessary, saccharide, dimethyl polysiloxane, and a digestive tract function-promoting agent) and has an osmotic pressure of 200 mOsm/L to 900 mOsm/L (preferably 200 mOsm/L to 570 mOsm/L, and more preferably 200 mOsm/L to 440 mOsm/L) by being suspended in a predetermined water can be prepared (Japanese Unexamined Patent Application, First Publication No. H4-259461), and a blister pack in which a liquid preparation or tablets including barium, an instruction in which the taking order is described, and a note in which symptoms of side effects and countermeasures are described are separately housed is detachably placed in the container. A form of inserting the plastic container with the blister pack in the container for suspending the composition for imaging a digestive tract or of attaching on the outer wall is useful. Convenience is further improved in a case where the container for suspending is filled with a liquid for suspending such as mineral water in advance.

In addition, the blister pack may be attached to a container filled with an intestinal tract cleaning liquid which contains an electrolyte and a water-soluble polymer (if necessary, saccharide, dimethyl polysiloxane, and a digestive tract function-promoting agent) and has an osmotic pressure of 200 mOsm/L to 900 mOsm/L (preferably 200 mOsm/L to 570 mOsm/L, and more preferably 200 mOsm/L to 440 mOsm/L).

As another example, a bag for kit in which each component and composition of the liquid preparation for oral administration B according to the present invention can be contained in three compartments formed by dividing a flexible plastic bag by the fused portion, to be referred to as a triple bag can be exemplified as a good example. The bag for kit has a feature that a liquid preparation for oral administration can be prepared by peeling the fused portion in the middle by pressing with hand or the like in use, and by mixing each component or composition which is a powder and the suspension thereof. Furthermore, in the case of printing an instruction and a note on the surface of this bag for kit, the bag can be used as a kit (petition 2000-2619).

In inflammatory bowel disease patients, there are concerns that the contrast agent stimulates inflammation of intestinal mucosa, and adversely effects on the diseased portion such as the ulcer portion, and thus, a steroid drug may be included in the kit or the packaged goods for the purpose of preventing inflammation.

[Drug for Pretreatment]

The drug for pretreatment according to the present invention is formed by combining barium and a water-soluble polymer or a saline purgative agent. In the drug for pretreatment, the barium and the water-soluble polymer or the saline purgative agent are respectively prepared as a separate liquid preparation, and are respectively taken all at once or in several parts over a period of time on the day before the CT colonography is performed. The total dose of the barium and the water-soluble polymer (or saline purgative agent) on the day before the CT colonography is performed is the same as in a case of being taken as the liquid preparation for oral administration B according to the present invention. That is, the total dose of the liquid preparation including the water-soluble polymer or saline purgative agent on the day before the CT colonography is performed is 50 mL to 2000 mL, preferably 50 mL to 1200 mL, more preferably 70 mL to 900 mL, still more preferably 100 mL to 9000 mL, and particularly preferably 100 mL to 450 mL. In addition, the total dose of a liquid preparation including barium on the day before the CT colonography is performed is preferably 6 g to 600 g, and more preferably 10 g to 100 g as barium sulfate.

The liquid preparation for oral administration B according to the present invention is a liquid preparation containing barium and a water-soluble polymer or a saline purgative agent, however, even in the case of separately taking a suspension of barium and a liquid preparation including a water-soluble polymer or a saline purgative agent respectively, it is possible to obtain the same effect as in the case of taking the liquid preparation for oral administration B according to the present invention if the total dose of the barium and the water-soluble polymer (or saline purgative agent) on the day before the CT colonography is performed is substantially the same as the total dose of the barium and the water-soluble polymer contained in the liquid preparation for oral administration B according to the present invention.

The liquid preparation including barium is preferably taken within at least 30 minutes before or after taking the liquid preparation including a water-soluble polymer or a saline purgative agent, and it is more preferable to be continuously taken. In addition, after taking a liquid preparation including barium, about 50 mL to 300 mL of water is preferably taken in.

In the case of taking a liquid preparation including barium and a liquid preparation including a water-soluble polymer or a saline purgative agent taken in several parts over a period of time, the number of times the liquid preparation including barium is taken and the number of times the liquid preparation including a water-soluble polymer or a saline purgative agent is taken may be the same or different. For example, the liquid preparation including a water-soluble polymer or a saline purgative agent and the liquid preparation including barium may be continuously taken three times a day, or only the liquid preparation including a water-soluble polymer or a saline purgative agent may be by dividing the dose into three times, and in one administration among these, the total amount of the liquid preparation including barium may be taken together with the liquid preparation including a water-soluble polymer or a saline purgative agent. As the liquid preparation including barium, a liquid preparation which includes barium sulfate in which the particle diameter D10 which is a diameter when the accumulation value in a volume cumulative distribution becomes 10% is 0.30 μm to 0.50 μm and the particle diameter D90 which is a diameter when the accumulation value value in a volume cumulative distribution becomes 90% is 1.00 μm to 2.00 μm, and has the viscosity at a shear rate of 21.54 s⁻¹ of 350 mPa·s or greater, and the viscosity at a shear rate of 464.1 s⁻¹ of 5 mPa·s to 90 mPa·s is preferable, and the liquid preparation for oral administration A is more preferable.

### EXAMPLES

Although the present invention will be described in further detail by showing the following Examples, the present invention is not limited to these.

### Example 1

The liquid preparation for oral administration which is intended to be taken in an amount of 50 mL to 1200 mL all at once or in several parts over a period of time on the day before examination by the CT colonography was performed was prepared as follows. First, one bag (137.155 g) of an oral intestinal tract cleaning agent (trade name: “Niflec (registered trademark)” mixed internal drug, manufactured by Ajinomoto Pharmaceuticals Co., Ltd.) was dissolved in water, whereby a solution of 2 L was obtained. 200 mL of an X-ray contrast agent (trade name: Bamstar S200, manufactured by Kyosei Pharmaceutical Co., Ltd.) for imaging the digestive tract including barium sulfate at a concentration of 200 w/v % was added to 1800 mL of the solution, whereby a liquid preparation for oral administration having a concentration of barium sulfate of 20 w/v % (0.2 g/mL) was prepared. Furthermore, in the same manner as in the preparation method described above, liquid preparations for oral administration having concentrations of barium sulfate of 2 w/v %, 5 w/v %, 8 w/v %, and 10 w/v % (0.02 g/mL, 0.05 g/mL, 0.08 g/mL, and 0.1 g/mL) were prepared.

### Example 2

By filling a flexible container made of polyethylene with 300 mL of the liquid preparation for oral administration having a concentration of barium sulfate of 10 w/v % (0.1 g/mL) prepared in Example 1 and by sealing the container, a liquid preparation for oral administration for taking in several times which could be taken in respectively 100 mL after breakfast, lunch, and dinner on the day before examination by the CT colonography was performed was prepared. The flexible container made of polyethylene filled with the liquid preparation for oral administration was enclosed in an outer packaging bag of a multilayer film formed of an aluminum deposited film having low gas permeability and sealed such that the concentration of sodium hydrogen carbonate included in the liquid preparation for oral administration does not change. Carbon dioxide gas was enclosed in the space between the flexible container made of polyethylene and the outer packaging bag along with air such that the concentration of sodium hydrogen carbonate included in the liquid preparation for oral administration does not change over time.

### Example 3

The liquid preparation for oral administration which is intended to be taken in an amount of 50 mL to 1200 mL all at once or in several parts over a period of time on the day before examination by the CT colonography was performed was prepared as follows. First, two bags (200 g) of a purgative agent for a large intestine examination or an abdominal surgery pretreatment (trade name: “Magcorol (registered trademark) P”, manufactured by Horii Pharmaceutical Ind., Ltd.) were dissolved in water, whereby a solution of 3600 mL was obtained. 200 mL of an X-ray contrast agent (trade name: Bamstar S200, manufactured by Kyosei Pharmaceutical Co., Ltd.) for imaging the digestive tract including barium sulfate at a concentration of 200 w/v % was added to 1800 mL of the solution, whereby a liquid preparation for oral administration having a concentration of barium sulfate of 20 w/v % (0.2 g/mL) was prepared. Furthermore, in the same manner as in the preparation method described above, liquid preparations for oral administration having concentrations of barium sulfate of 2 w/v %, 5 w/v %, 8 w/v %, and 10 w/v % (0.02 g/mL, 0.05 g/mL, 0.08 g/mL, and 0.1 g/mL) were prepared.

### Example 4

100 mL of the solution of the oral intestinal tract cleaning agent [solution of 2 L obtained by dissolving one bag (137.155 g) of an oral intestinal tract cleaning agent (trade name: “Niflec (registered trademark)” mixed internal drug, manufactured by Ajinomoto Pharmaceuticals Co., Ltd.) in water] and 8 mL (14.2 g) of an X-ray contrast agent (trade name: “Barytgen” (registered trademark) sol, manufactured by Fushimi pharmaceutical Co., Ltd.) for the digestive tract containing barium sulfate of 100 w/v % were administered to a man of 50 years of age after each meal of breakfast, lunch, and dinner (without particular dietary restriction) on the day before examination by the CT colonography was performed. Although 260 mL of water was inevitably taken in after barium was taken, the others were freely taken in. The amount of water that the subject took on the day before examination was performed was about 2 L including the water taken in after taking barium (the amount of 260 mL×3 times). On the examination day of the CT colonography, the subject fasted until the examination ended.

The meal on the day before examination by the CT colonography was performed was a normal meal rather than the so-called examination meal, except for being careful so as not to take in the meal including vegetables and fruits (except for a paste and a juice) of which the form remains, seaweeds (wakame, kelp, hijiki, and the like), mushrooms (shiitake mushroom, shimeji mushroom, enoki mushroom, and the like), beans (green soybean, black soybean, and the like), shirataki, and konjak. The subject excreted solid stool before the examination on the examination day, the stool was white-grayish brown, and barium seemed to be substantially uniformly distributed.

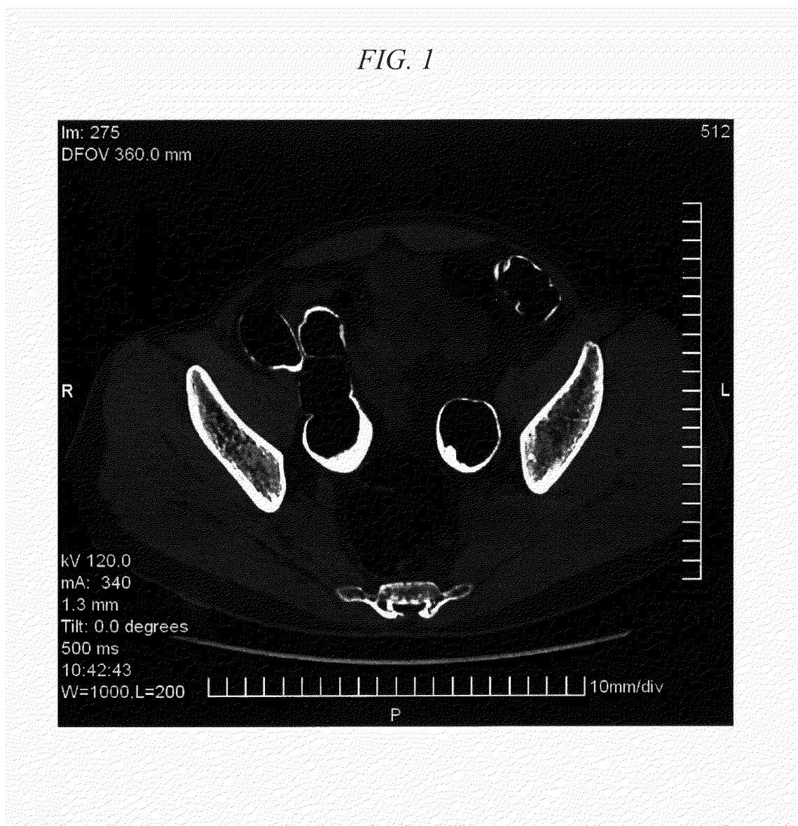

Carbon dioxide gas was injected per anum, and CT images were captured for supine and prone positions by using a 128-slice CT apparatus HDCT VEO type (manufactured by GE Healthcare Corporation). Although it was expected that stool mass remained in the large intestine from the fact that there was excretion of solid stool on the examination day, there was no problem in supply of carbon dioxide, and carbon dioxide could be smoothly supplied. FIG. 1 shows a CT image of a supine position of a descending colon. As shown in FIG. 1, even in the descending colon, adhesion of barium was observed on a part of the intestinal tract wall, however, the residues were substantially homogeneously labeled with barium, and visually, the tissues and the stool mass or the stored residues could be identified. That is, it was found that even in a case where excretion was not forcibly performed by a laxative or the like, the CT colonography was executable by performing a pretreatment using the drug for pretreatment for the CT colonography according to the present invention.

Moreover, in the Example, after 8 mL of barium sulfate was taken, 100 mL of a solution of an oral intestinal tract cleaning agent was quickly taken, and then, 260 mL of water was taken in, however, even in a case where a mixture obtained by mixing 100 mL of a solution of an oral intestinal tract cleaning agent and 8 mL of barium sulfate in advance was taken, and then, 260 mL of water was taken in, similar results were obtained.

### Example 5

<Preparation of Barium Prototype Liquid Preparation>

First, in order to administer on the day before examination by the CT colonography was performed, a liquid preparation for oral administration containing two types of barium sulfates having different flow characteristics was prepared.

Specifically, barium sulfate having the particle diameter D10 of 0.39 μm and the particle diameter D90 of 1.35 μm was dispersed in aqueous solutions in which a natural polysaccharide and carboxymethyl cellulose were contained, whereby two types of barium prototype liquid preparations having a concentration of barium sulfate of 0.25 g/mL were prepared. The flow characteristics of these barium prototype liquid preparations a and b were prepared so as to have the values shown in Table 1 by suitably adjusting the concentration of the natural polysaccharide and the concentration of the carboxymethyl cellulose.

TABLE 1

Viscosity (mP · s)

| Shear | Barium prototype | Barium prototype |
|---|---|---|
| rate (1/s) | liquid preparations a | liquid preparation b |
| 21.54 | 371.8 | 11900 |
| 464.1 | 41.57 | 1057 |

Moreover, the particle size distribution of barium sulfate was measured using a laser diffraction scattering type particle size distribution measuring apparatus “Microtrac MT3300EX2” (manufactured by Nikkiso Co., Ltd.). After a sample (about 0.45 μL) was mixed with 2 mL of 2% by mass sodium hexametaphosphate solution, built-in ultrasonic waves were applied for 3 minutes, and the particle size distribution was measured.

In addition, the viscosity of the barium prototype liquid preparation at a shear rates of 21.54 s⁻¹ and 464.1 s⁻¹ was measured using a cone-plate type rotational viscometer “MCR300” (manufactured by Anton Paar GmbH). Specifically, the viscosity of a sample (1.2 mL) was measured while continuously changing the shear rate within a range of 0.01 s⁻¹ to 1000 s⁻¹ at 25° C. using a cone plate “CP50-2” (thirty one points were measured).

100 mL of the solution of the oral intestinal tract cleaning agent [solution of 1800 mL obtained by dissolving one bag (100 g) of a purgative agent for an large intestine examination or an abdominal surgery pretreatment (trade name: “Magcorol (registered trademark) P”, manufactured by Horii Pharmaceutical Ind., Ltd.) in water] and 40 mL of the barium prototype liquid preparation a were administered to a man of 40 years of age after each meal of breakfast, lunch, and dinner on the day before examination by the CT colonography was performed. Although 260 mL of water was inevitably taken in after the barium prototype liquid preparation a was taken, when the other was freely taken in, the amount of the water that the subject took on the day before examination was performed was about 2 L including the water taken in after taking barium (the amount of 260 mL×3 times). On the day when examination by the CT colonography was performed, the subject fasted until the examination ended. The subject excreted loose stool before the examination on the examination day, the stool was white-grayish brown, and barium seemed to be substantially uniformly distributed.

Moreover, the meal on the day when examination by the CT colonography was performed was the so-called examination meal (curry and rice, bowl of rice with chicken and eggs, and corn soup). The examination meal was a meal which was cooked so as to be the same meal as in a normal diet except that taking in of the meal including vegetables and fruits (except for a paste and a juice) of which the form remained, seaweeds (wakame, kelp, hijiki, and the like), mushrooms (shiitake mushroom, shimeji mushroom, enoki mushroom, and the like), beans (green soybean, black soybean, and the like), shirataki, and konjak was avoided.

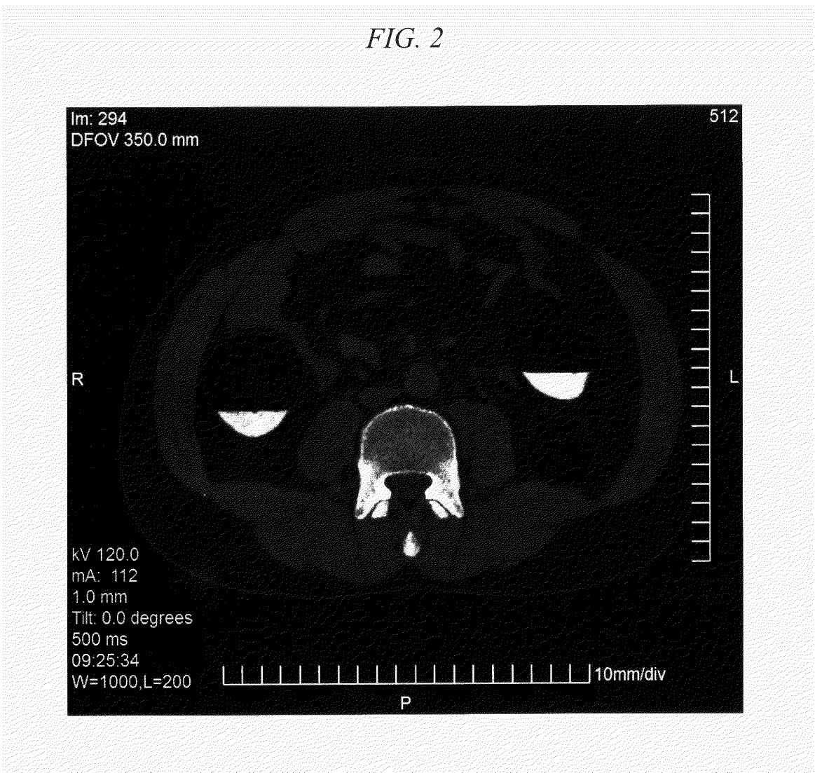

Carbon dioxide gas was injected per anum, and CT images were captured for supine and prone positions by using a 64-row multislice CT apparatus “Toshiba scanner Aquilion (registered trademark) TSX-101A” (manufacture by Toshiba Medical Systems Corporation). FIG. 2 shows a CT image of a supine position of a descending colon. As shown in FIG. 2, even in the descending colon, adhesion of barium was not observed on the intestinal tract wall, the residues were homogeneously labeled with barium, and visually, the tissues and the stool mass or the stored residues could be identified. In addition, the residues were not a solid but a uniformly labeled liquid, and thus, excellent CT images in which the intestinal tract inner wall was clearly expressed were obtained. The reason for this was that, in the case of liquid residues, the residues moved by the change of position, and thus, it was possible to treat in the same manner as the case of completely removing the residues virtually. That is, it was found that by performing a pretreatment using the liquid preparation for oral administration B according to the present invention, the residues due to a meal can be uniformly labeled, and even in a case where excretion was not forcibly performed by a laxative or the like, the CT colonography was excellently executable.

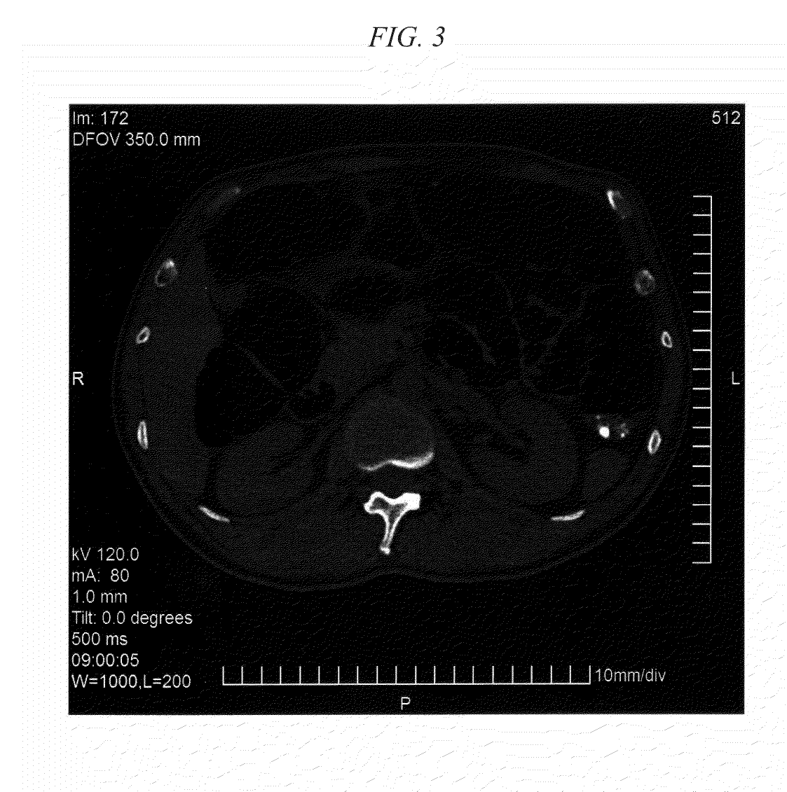

In the same manner except for using the barium prototype liquid preparation b instead of the barium prototype liquid preparation a, the solution of the oral intestinal tract cleaning agent and the barium prototype liquid preparation were taken on the day before the CT colonography examination was performed, and the examination was performed. The result of the CT imaging is shown in FIG. 3. As shown in FIG. 3, in the case of using the barium prototype liquid preparation b, the barium in the remaining water in the intestine was precipitated.

### Example 6

In the intestinal tract, the barium solution taken was present in a diluted state by body fluids or water taken in at the same time. Accordingly, for the barium prototype liquid preparation a prepared in Example 5, the ease of precipitation in a standing state after dilution was examined. Measurement was performed on barium prototype liquid preparations c and d having the particle diameter D10 and the particle diameter D90 of the values shown in Table 2 and having the viscosities shown in Table 1 as an object of comparison in the same manner. The particle size distribution and the viscosity of the barium prototype liquid preparations c and d were measured in the same manner as in Example 5.

TABLE 2

| | Barium prototype | Barium prototype |
|---|---|---|
| | liquid preparation c | liquid preparation d |
| Particle diameter D10 (μm) | 0.46-0.5 | 0.63 |
| Particle diameter D90 (μm) | 2.6-2.9 | 2.4 |

TABLE 3

Viscosity (mP · s)

| Shear | Barium prototype | Barium prototype |
|---|---|---|
| rate (1/s) | liquid preparation c | liquid preparation d |
| 21.54 | 170-210 | 310-330 |
| 464.1 | 68-80 | 45-49 |

The dose (24 g in terms of barium sulfate) of one day of each barium sample (barium prototype liquid preparations a, c, and d) was added to water to prepare a suspension (concentration of barium sulfate: 12 g/L) adjusted to 2 L. 100 mL of this suspension was dispensed to each 100 mL graduated cylinder, and the resultant products were allowed to stand at room temperature for 12 hours. After standing, from the scale of the graduated cylinder, the amount of supernatant (the amount which became transparent due to precipitation of barium sulfate) was measured. The measurement results are shown in Table 4 along with the liquid volume of each barium sample required to prepare 2 L of an aqueous solution having a concentration of barium sulfate of 12 g/L (in the table, “added amount”).

In addition, a suspension (concentration of barium sulfate: 12 g/L) was prepared by dissolving a daily dose (24 g in terms of barium) of one day of each barium sample (barium prototype liquid preparations a, c, and d) and one bag (137.155 g) of the oral intestinal tract cleaning agent (trade name: “Niflec (registered trademark)” mixed internal drug, manufactured by Ajinomoto Pharmaceuticals Co., Ltd.) in water and by adjusting to be 2 L. This suspension was dispensed by 100 mL to each of 100 mL graduated cylinders, the resultant products were allowed to stand at room temperature for 12 hours, and from the scale of the graduated cylinder, the amount of supernatant (amount which became transparent due to precipitation of barium) was measured. The measurement results are shown in Table 4.

TABLE 4

| | | Amount of | Amount of |
|---|---|---|---|
| | Added | supernatant | supernatant |
| | amount | (water) | (Niflec) |
| Barium prototype | 96 mL | 1 mL | 1 mL |
| liquid preparation a | | | or less |
| Barium prototype | 24 mL | 5 mL | 5 mL |
| liquid preparation c | | or greater | |
| Barium prototype | 60 mL | 5 mL | 3 mL |
| liquid preparation d | | or greater | |

It was found that, in any barium sample, little precipitation of barium was observed even after standing for 12 hours in a diluted state, and thus, even in a diluted state in the intestinal tract, barium was less likely to precipitate, and the remaining water can be substantially uniformly labeled. In particular, it was found that the barium prototype liquid preparation A had the amount of supernatant clearly less than any commercial liquid preparation, and the dispersion stability of the barium in a standing state was excellent. In addition, it was found that the amount of supernatant in the case of diluting with the oral intestinal tract cleaning agent was less than that in the case of diluting with water, and thus, when taking the liquid preparation for oral administration A or B according to the present invention as a pretreatment for the CT colonography, it was preferable to take with the oral intestinal tract cleaning agent such as Niflec than with water.

### INDUSTRIAL APPLICABILITY

The liquid preparation for oral administration and the like according to the present invention can be suitably used in the pretreatment for the CT colonography, and can be used in the field of clinical examination such as a large intestine examination, in particular, in the primary screening examination such as a mass examination.

1. A liquid preparation for oral administration, comprising:
barium sulfate; and
one or more types of thickening agents selected from the group consisting of a natural polysaccharide and a cellulose-based polymer,
wherein the viscosity at a shear rate of 21.54 s⁻¹ is 100 mPa·s or greater and the viscosity at a shear rate of 464.1 s⁻¹ is 5 mPa·s to 90 mPa·s, and
wherein a particle diameter D10 which is a diameter when the accumulation value in a volume cumulative distribution of the barium sulfate becomes 10% is 0.30 μm to 0.80 μm and a particle diameter D90 which is a diameter when the accumulation value in a volume cumulative distribution of the barium sulfate becomes 90% is 1.0 μm to 10 μm.

2. The liquid preparation for oral administration according to claim 1, wherein the concentration of the barium sulfate is 0.15 g/mL to 0.35 g/mL.

3. The liquid preparation for oral administration according to claim 1, wherein the viscosity at a shear rate of 21.54 s⁻¹ is 350 mPa·s or greater.

4. The liquid preparation for oral administration according to claim 1, wherein the particle diameter D10 which is a diameter when the accumulation value in a volume cumulative distribution of the barium sulfate becomes 10% is 0.30 μm to 0.50 μm and the particle diameter D90 which is a diameter when the accumulation value in a volume cumulative distribution of the barium sulfate becomes 90% is 1.00 μm to 2.00 μm.

5. The liquid preparation for oral administration according to claim 1, wherein the thickening agent is one or more types selected from the group consisting of guar gum, carrageenan, xanthan gum, fenugreek gum, pectin, pullulan, gum arabic, dextran, dextrin, polydextrose, hydroxyethyl starch, carboxymethyl cellulose and salts thereof, hydroxypropyl cellulose, gum tragacanth, sodium alginate, sodium chondroitin sulfate, locust bean gum, gellan gum, croscarmellose sodium, and crystalline cellulose-carmellose sodium.

6. The liquid preparation for oral administration according to claim 1, which is used for imaging of a digestive tract on CT colonography.

7. A liquid preparation for oral administration, which is used for imaging a digestive tract on CT colonography digestive tract imaging and is taken in an amount of 50 mL to 2000 mL all at once or in several parts in over a period of time on the day before CT colonography is performed, comprising:
barium compound; and
a water-soluble polymer or a saline purgative agent.

8. The liquid preparation for oral administration according to claim 7, which is taken in an amount of 70 mL to 900 mL all at once or in several parts in the day before the CT colonography is performed.

9. The liquid preparation for oral administration according to claim 7, wherein the barium compound is barium sulfate.

10. The liquid preparation for oral administration according to claim 7, wherein the water-soluble polymer is one or more types selected from the group consisting of polyethylene glycol, polydextrose, dextran, dextrin, hydroxyethyl starch, gum arabic, pullulan, pectin, albumin, carboxymethyl cellulose and salts thereof, carrageenan, xanthan gum, gum tragacanth, croscarmellose sodium, and crystalline cellulose-carmellose sodium.

11. The liquid preparation for oral administration according to claim 7, wherein the osmotic pressure is 200 mOsm/L to 900 mOsm/L.

12. The liquid preparation for oral administration according to claim 7, further comprising:
one or more types of saccharides selected from the group consisting of sucrose, sorbitol, xylitol, erythritol, mannitol, trehalose, lactitol, lactulose, maltitol, palatinose, raffinose, and glycerin.

13. The liquid preparation for oral administration according to claim 7, wherein the amount of the barium compound is 8.3 mg/mL to 2000 mg/mL.

14. The liquid preparation for oral administration according to claim 7, wherein the concentration of the barium compound as barium sulfate is 0.15 g/mL to 0.35 g/mL.

15. The liquid preparation for oral administration according to claim 14, wherein the viscosity at a shear rate of 21.54 s⁻¹ is 350 mPa·s or greater, and the viscosity at a shear rate of 464.1 s⁻¹ is 5 mPa·s to 90 mPa·s.

16. The liquid preparation for oral administration according to claim 7, wherein a particle diameter D10 which is a diameter when the accumulation value in a volume cumulative distribution of the barium becomes 10% is 0.30 μM to 0.50 μm and a particle diameter D90 which is a diameter when the accumulation value in a volume cumulative distribution of the barium becomes 90% is 1.00 μm to 2.00 μm.

17. The liquid preparation for oral administration according to claim 7, further comprising:
one or more types selected from the group consisting of dimethyl polysiloxane and a digestive tract function-promoting agent.

18. A composition for imaging a digestive tract, which is dissolved in water taken in and an amount of 50 mL to 2000 mL all at once or in several parts over a period of time in the day before CT colonography is performed, as an aqueous solution in which the amount of the barium compound is 8.3 mg/mL to 2000 mg/mL obtained by being dissolved in water, comprising:
barium compound; and
a water-soluble polymer or a saline purgative agent.

19. The composition for imaging a digestive tract according to claim 18, which is to be dissolved in water and taken in an amount of 70 mL to 900 mL all at once or in several parts in the day before the CT colonography is performed, as an aqueous solution in which the amount of the barium compound is 8.3 mg/mL to 2000 mg/mL obtained by being dissolved in water.

20. The composition for imaging a digestive tract according to claim 18, wherein the barium compound is barium sulfate.

21. The composition for imaging a digestive tract according to claim 18, further comprising:
an electrolyte by which the osmotic pressure becomes 200 mOsm/L to 900 mOsm/L when dissolved in water; and
one or more types of water-soluble polymers selected from the group consisting of polyethylene glycol, polydextrose, dextran, dextrin, hydroxyethyl starch, gum arabic, pullulan, pectin, albumin, carboxymethyl cellulose and salts thereof, carrageenan, xanthan gum, gum tragacanth, croscarmellose sodium, and crystalline cellulose-carmellose sodium, or one or more types of saline purgative agents selected from the group consisting of magnesium citrate, sodium dihydrogen phosphate, and disodium hydrogen phosphate.

22. The composition for imaging a digestive tract according to claim 18, further comprising:
one or more types selected from the group consisting of dimethyl polysiloxane and a digestive tract function-promoting agent.

23. The composition for imaging a digestive tract according to claim 18, wherein a particle diameter D10 which is a diameter when the accumulation value in a volume cumulative distribution of the barium becomes 10% is 0.30 μm to 0.50 μm and a particle diameter D90 which is a diameter when the accumulation value in a volume cumulative distribution of the barium becomes 90% is 1.00 μm to 2.00 μm.

24. The composition for imaging a digestive tract according to claim 23, which is in liquid form, wherein the viscosity at a shear rate of 21.54 s⁻¹ is 350 mPa·s or greater, and the viscosity at a shear rate of 464.1 s⁻¹ is 5 mPa·s to 90 mPa·s.

25. A drug for pretreatment for CT colonography, composed of barium compound and either of water-soluble polymer or saline laxatives, wherein the barium compound and each of 50 mL to 2000 mL of an aqueous solution obtained by dissolving the water-soluble polymer or the saline purgative agent in water and the barium are taken all at once or in several parts in the day before the CT colonography is performed.

26. The drug for pretreatment for CT colonography according to claim 25, wherein the water-soluble polymer or the saline purgative agent dissolved in water and the barium are taken within 30 minutes.

27. The drug for pretreatment for CT colonography according to claim 25, wherein the total dose of the aqueous solution on the day before the CT colonography is performed is 70 mL to 900 mL.

28. The drug for pretreatment for CT colonography according to claim 25, which is a liquid preparation in which the barium compound is barium sulfate in which a particle diameter D10 which is a diameter when the accumulation value in a volume cumulative distribution becomes 10% is 0.30 μm to 0.50 μm and a particle diameter D90 which is a diameter when the accumulation value in a volume cumulative distribution becomes 90% is 1.00 μm to 2.00 μm, and the viscosity at a shear rate of 21.54 s⁻¹ is 350 mPa·s or greater, and wherein the viscosity at a shear rate of 464.1 s⁻¹ is 5 mPa·s to 90 mPa·s.

29. A liquid preparation for oral administration, which is used for imaging a digestive tract on in CT colonography digestive tract imaging, and is taken barium in an amount of 50 mL to 2000 mL with or without barium compound all at once or in several parts in the day before the CT colonography is performed, comprising:
a water-soluble polymer or a saline purgative agent.
